(19) United States
(12) Patent Application Publication
(10) Pub. No.: US 2002/0076444 A1
(43) Pub. Date: Jun. 20, 2002

(54) NOVEL METHOD FOR OBTAINING MICROSPHERES AND RESULTING PRODUCTS

(76) Inventors: FRANCIS DI COSTANZO, PARIS (FR); JACQUELINE J. DI COSTANZO, PARIS (FR); GERARD COUSIN, LE MESNIL-PONCEAU (FR); ETIENNE BRUNA, JOUY (FR); FRANCOISE RAGOT, LEVES (FR); JEAN-PHILIPPE AURIAT, PARIS (FR); JULIEN MEISSONNIER, CAEN (FR)

Correspondence Address: BIERMAN MUSERLIAN AND LUCAS, 600 THIRD AVENUE, NEW YORK, NY, 10016

(21) Appl. No.: 09/331,465

(22) PCT Filed: Oct. 21, 1998
(86) PCT No.: PCT/FR98/02251

(30) Foreign Application Priority Data
Oct. 21, 1997 (FR) 97/13155
Oct. 20, 1998 (FR) 98/13150

(51) Int. Cl. A61K 9/14

(52) U.S. Cl. 424/489

(57) ABSTRACT

The invention concerns the field of therapeutic or food chemistry, and more particularly the production of novel galenic forms. It concerns a novel method for obtaining microspheres with predetermined particle size distribution, containing an active principle using a matrix-forming agent made of a hydroalkane solution in the presence of a surfactant, to form an O/W emulsion of said matrix-forming agent, which consists in allowing the alkanol from the emulsion droplets to be diffused towards the continuous phase, so as to form in the droplet a coprecipitate of matrix-forming agent and active principle, in separating the microspheres, in washing and drying them to collect solid or hollow microspheres. The invention is useful in therapy, for dyes, in the food industry and as flavoring agents.

The present invention relates to the area of therapeutic or alimentary chemistry and more especially to the production of new galenic forms.

The subject-matter of the invention relates more particularly to the production of solid or hollow microspheres intended to receive pharmaceutical active principles, alimentary or chemical products located in the matrix or in the walls of the latter.

The subject-matter of the invention specifically relates to a new process for obtaining microspheres with a determined granulometric distribution containing an active principle by using a matrix-forming agent in a hydroalkanolic solution in the presence of a surfactant, by forming an O/W emulsion of this matrix-forming agent, by allowing the alkanol to diffuse from the emulsion droplets towards the continuous phase in such a way as to form in the droplet a co-precipitate of a matrix-forming agent and active principle, in separating the microspheres, washing them and drying them so as to recover solid microspheres.

The invention also relates to a process for obtaining hollow microspheres in which the water-insoluble matrix-forming agent and the active principle are dispersed in an alkanol, then it is mixed with water to form an O/W emulsion divided in droplets in a continuous aqueous phase, the alkanol is allowed to diffuse towards the aqueous phase, to ensure the solidification of the wall of the droplet, then the microspheres are separated, washed and dried in such a way that hollow microspheres are recovered.

In this process, the insoluble matrix-forming polymer is in particular an acrylic ester polymer, neutral or charged, ionic or not, for example acrylic acid polymers carrying a quaternary ammonium group having the formula:

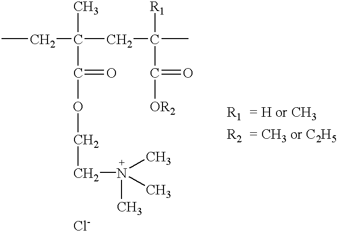

They are marketed in particular by the firm Röhm Pharma GmbH under the names Eudragit RS, Eudragit RL, Eudragit S, Eudragit L, Eudragit E, Eudragit NE.

These polymers have already been widely used in the literature as a polymeric coating material for the development of microcapsules and microspheres (S. Benita, J of Controlled Release 12 (1990) 213). The advantage of acrylic polymers is that they are able to mask the taste of the active principle and that they permit a controlled release of the latter.

The microsphere technique has its origin in the search for pharmaceutical forms with a controlled release by producing forms that are not attacked or disintegrated in the gastric medium One of the first attempts thus consisted in producing empty globular shells having a density less than that of the gastric or intestinal juice in such a way as to permit the shell to float at the surface of the juice and to permit a longer dwell time in the stomach. One such type of formulation was described initially with inert polymers such as polystyrene then with mixtures of hydrocolloids which upon contact with the gastric or intestinal juices swell and form a smooth gelatinous mass at the surface (Y. Kawashima, Int. J of Pharmaceutics 75 (1991) 25). This kind of particle possesses a lower density and floats at the surface of the gastric or intestinal juice.

Later on, it was further recommended to use microspheres formed from chemically inert but biodegradable polymers such as polylactides (Wakiyama, Chem Parm Bull. 30 (1982) 2621) or polyglycolic acids (T. Sato Pharm Research 5 (1988) 21) or polylactide/polyglycolide mixtures (Sanders J. Pharm Sci. 73 (1984) 1294).

The microsphere technique in fact assumed its outstanding importance with the work carried out by Y. Kawashima, J Pharm. Sci. 73 (1984) 1535 which made available the so-called spherical crystallisation technique during crystallisation, by using a binary or ternary mixture of solvents, some being hydrophilic (ethanol) the others lipophilic (chloroform or dichloromethane).

This technique was developed by using polyacrylates as matrix-forming polymers at the surface of the emulsion droplet, in such a way as to reduce the rate of dissolution and to present a uniform surface covered by the polymer. Micropores of small dimensions (diameter <10μm) appear at the surface of the microspheres, especially when the latter are prepared with weak polymer concentrations.

To produce microspheres exhibiting a continuous surface, having a determined porosity, a suitable mechanical strength and a controlled release time of the active principle, a large number of factors have to be taken into account.

One of the important factors is the rate of diffusion of the solvent from the O/W emulsion droplet towards the aqueous phase at the initial stage. According to the work of Y. Kawashima (J.Micro Encapsulation 10 (1993) 329), this factor is decisive. If the rate of diffusion of the solvent is too rapid, the droplets of O/W emulsion could not be formed. On the other hand, an excessively slow diffusion of the solvent leads to the agglomeration of microspheres during the treatment. Accordingly, the literature states that microspheres containing ketoprofen, could not be produced by micro-encapsulation by the method of diffusion of the solvent in emulsion (employing ethanol as the solvent) due to the fact that the slow diffusion of the solvent leads to marked agglomeration.

This is the reason why previous experimenters have generally incorporated in the solvent a surfactant of the sucrose-ester type to facilitate the diffusion of the solvent and to avoid agglomeration of micro particles.

The Applicants have found that, for the purpose of forming microspheres, the use of a volatile organic solvent inmiscible with water, such as a halogenated solvent and in particular chloroform or methylene chloride, was not essential and that it was still possible to produce at will, solid or hollow microspheres with a determined granulometry, by using the following parameters:

the ratio and the type of matrix-forming polymer/pharmaceutical active principle, alimentary or chemical product,

the duration, position and mode of incorporation of the organic phase,

the nature and the quantity of surfactant,

the nature and the quantity of solvent used,

the speed, time and type of stirring,

the method of filtering the particles,

the temperature and the type of drying,

the working temperature for the two phases,

the adjustment of the pH value as a function of the chemical nature of the active principle to be incorporated,

the adaptation of a suitable viscosity of the aqueous phase (alginate, PVP).

The two types of particles, and more particularly the solid particles, are suitable for producing essentially tasteless formulations containing an active principle with an unpleasant taste, where it will essentially be hidden. Thus, the advantage of these two forms is that they can be compacted, and thus lead to various galenic forms permitting the controlled, immediate or prolonged release of a pharmaceutical active principle, an alimentary or chemical product. These solid particles find a particular use in tablets with rapid disintegration in the mouth of the Flashtab® type as described in French patent 2.679.451.

It was particularly noted that this process was dependent on two parameters that are more particularly important, the quantity of dry matter provided by the matrix-forming polymer and the volume of solvent required to disperse this polymer. An increase in the ratio mass of dry matter/volume of solvent leads to an increase in the porosity of the particles. In addition, an increase in the quantity of matrix-forming polymer, in particular of cellulose derivatives, leads to the formation of cavities in the particles.

Moreover, proceeding from a system permitting hollow particles to be obtained, makes possible, by raising the temperature of the aqueous phase, to obtain solid particles and vice versa. It is thus possible to manufacture at will solid or hollow microspheres.

Furthermore, it was found that the type of surfactant plays an important role in the appearance of the particles.

Equally, the speed, position and type of stirring, as well as the mode and rate of incorporation, play a very important role in the granulometry of the particles. It is also possible, by incorporating an inert diluting agent in the organic phase, to produce more compact microsphere walls which ensure a better masking of the taste of the active ingredient in the microspheres or a greater mechanical strength of the microspheres.

It is thus possible to ascertain the advantage and the extensive nature of the process of producing microspheres according to the invention. It is also possible to envisage the use of cross-linking products as a hardening agent (tannin, formol).

The choice of a determined matrix-forming polymer, the nature of the surfactant, the stirring speed and the quantity of matrix-forming polymer, the M/v ratio (mass of dry matter/volume of alkanol), the percentage of polymer as well as the temperature and the volume of organic solvent allow one to choose between the production of solid microspheres, hollow microspheres, and particles with variable porosity, or to produce solid microspheres from hollow microspheres or vice versa, by acting on the temperature of the experiment. The process thus shows that there is a common production stem which can lead directly or indirectly to several types of production whose useful purposes are different.

The process such as described also has the advantage that it is possible to incorporate in the solid or hollow microspheres any type of drug, flavour, aroma, chemical product, whose release can be predetermined either to achieve an immediate release, or a prolonged release when it is desired to control the release of the active agent or agents over time.

Furthermore, the process according to the invention allows the microspheres to be coated with inert agents thus leading to products which are less porous and which will be permeated by aqueous liquids in a much more prolonged fashion.

The invention thus-presented, therefore permits the incorporation in microspheres of solid, liquid or oily products as defined in the examples, in such a way as to produce more easily manageable solid pharmaceutical forms of liquid or oily products, to protect fragile products, to stabilise and/or preserve flavours or aromatic products and to ensure the preservation of chemical or alimentary products fragile in light and/or in atmospheric oxygen.

The invention further relates to the use of microspheres according to the invention as a form with controlled release of active principles.

Hollow microspheres loaded with an active principle, particularly a pharmaceutical one, can be designed to be lighter than the gastric juice and to float at its surface. In this way, the attack by the gastric juice is prolonged.

Finally, by depositing an inert diluting agent or a film-forming agent on the moist microspheres, a functional coating is formed after drying, and a longer or shorter delay in the release of the active principle is thus achieved. For this purpose, it is possible to employ coating in a fluidised air bed or by coacervation. Inert diluting agent is understood to mean any mineral or organic matter that does not enter into reaction, such as colloidal silica, talc, insoluble stearates, polyethylene glycols and polyethylene glycol stearates.

The microspheres according to the invention can also be used as carriers for drugs or active principles sparingly soluble in the aqueous phase but soluble in the organic solvent. In this regard, mention can be made of steroid hormones such as estradiol or progesterone; phenyl-acetic or phenyl-propionic acid derivatives such as ketoprofen, tiaprofenic acid or indomethacin; ergot alkaloid derivatives antihistaminic agents such as tranilast, ibudilast or tazanolast; iodinated contrast agents; aspirin antibiotics; nitrated anticoccidial agents; agents exerting an effect on blood coagulability, anti-epileptic drugs; myorelaxants, drugs used in the treatment of diabetes, drugs used in the treatment of thyroid dysfunctions, diuretics, anorexiants, anti-asthmatic drugs, expectorants, antitussives, mucoregulators, decongestants, hypnotics, antinauseants, haematopoietic agents, uricosurics, vegetable extracts, contrast agents.

The microspheres according to the invention can also incorporate medicinal active ingredients having a solubility in water, including gastrointestinal sedatives, antacids, antalgics, anti-inflammatory drugs, coronary vascodilators, peripheral and cerebral vasodilators, anti-infective drugs, antibiotics, antiviral drugs, anti-parasitic drugs, anticancer drugs, anxiolytic drugs, neuroleptics, stimulants of the central nervous system, antidepressants, antihistaminics, anti-diarrhoeal drugs, laxatives, nutritional supplements, immunodepressants, hypocholesterolemiants, hormones, enzymes, antispasmodics, anti-angina drugs, drugs influencing cardiac rhythm, drugs used in the treatment of arterial hypertension, anti-migraine drugs, anti-coagulants, antithyroid agents, diuretics, anti-diuretics, anorexiants, anti-asmathic drugs, expectorative agents, antitussive agents, mucoregulators, nasal decongestants, hypnotic drugs, antinausea drugs, gastric and/or intestinal mobility modifiers, anti-anaemic drugs, uricosuric agents and/or contrast agents.

The thickness of the microsphere wall or the diameter of the matrix regulated by an efficient concentration of matrix polymer as well as the type of polymer constitute the important parameter for determining the rate of release of the active principle. In the process according to the invention, the content of active ingredient incorporated in the microspheres may range from 1 to 99% in the dry product with a preference for contents ranging from 4 to 95%, and more particularly from 50 to 95%.

The alkanol used to solubilise the active principle and the matrix-forming polymer is a lower alkanol preferably miscible with water, in particular ethanol, isopropanol, butanol or terbutanol. The concentration of alkanol can range between 0.5 and 20%, preferably from 0.5 to 15%.

The matrix polymer can also be a cellulose polymer such as for example the cellulose ethers or esters, the methacrylates, the polymethacrylamides, the EVA copolymers (ethylene/vinyl acetate), modified glucides such as for example starches cross-linked by epichlorhydrin, polyvinyl pyrrolidones, polyvinyl polypyrrolidones or cross-linked polyvinyl pyrrolidones. By way of example, mention may be made of hydroxyethyl cellulose, hydroxypropyl cellulose, hydroxypropylmethyl cellulose, polylactide, polyglycolic acid or polylactide/polyglycolide mixtures.

The content of matrix-forming polymer incorporated in the microspheres ranges from 1 to 99%, preferably from 5 to 80% in the dry product.

The surfactant incorporated in the aqueous phase is a non-ionic surfactant such as for example a Tween, a Span or a Brij or an anionic surfactant such as for example sodium lauryl-sulphate, sodium dodecyl-sulphate or sodium docusate, or a cationic surfactant, like the quaternary ammonium salts such as cetalkonium chloride, cetrimonium bromide, cetrimonium stearate or benzalkonium chloride. The content of each surfactant ranges from 0 to 20% in the dry product and preferably from 0.1 to 11% in the aqueous phase, but reaches higher values in the organic phase. Use is preferably made of a non-ionic surfactant such as the poloxameres or the poloxalenes (block copolymers of the type ethylene oxide/propylene oxide).

Depending on the system, the rate of incorporation will range between 1 second and 30 minutes, and preferably between 1 second and 4 minutes.

The stirring of the emulsion is an important factor and takes place for a period ranging from 1 to 120 minutes at a stirring speed ranging from 100 to 10,000 revs/min, and preferably for 2 to 45 minutes.

For example, it is possible to modify the size, the consistency of the microspheres and the time of release of the active principle that is contained therein. Thus it is that, for one and the same formula (percentage of solution, percentage by volume, . . . ), Eudragit RS 100 provides small very porous microspheres whereas Eudragit RL 100 provides under the same conditions large very compact and only slightly porous microspheres. The association of two acrylates (RL 100/RS 100=4/3) leads to the formation of solid, small and compact particles releasing in vitro at least 85% of the active principle in 45 minutes.

The filtration of the microspheres takes place by suction for example on a fritted glass sheet or on a filtering funnel or by centrifugation by using for example a Guinard or Rousselet centrifuge and in a general manner by using filtration equipment commonly used in industry.

The drying is generally carried out at a temperature varying from 25 to 100 C. and preferably between 40 and 70 C. The drying time varies according to the temperature, the apparatus used, and also depending on the type of drier used. The duration of drying the microspheres between 1 and 48 hours contributes notably to the physical properties of the matrix. It will thus be able to be carried out in a rotatory drier under vacuum of the Klein type, or with a fluidised air bed of the Glatt type, or again any commonly used industrial equipment.

In conclusion, it emerges that the thus-defined invention permitting the production of solid and/or hollow microspheres offers the advantage of being easy to use, of obtaining hollow microspheres without chlorinated solvent for the two types of microspheres, of a satisfactory appearance, of good reproducibility, of a granulometry suitable either for masking the taste of certain molecules or for the incorporation of active ingredients in a soluble form and furthermore for being able to control the time of dissolution of the microspheres of both types or to complement the regulation of this release with the aid of a specific coating.

Supplementary studies have also demonstrated the flexibility of the process and the possibility of incorporating a very wide variety of active principles.

In the case of hollow microspheres, the solvent miscible with the aqueous phase undergoes a counter-diffusion towards the aqueous phase. In this way, only a single phase remains inside the cavities of the particle, such that the washing and drying lead to complete evaporation of the aqueous phase contained in the microsphere, and leave an internal cavity more or less filled with air.

A further feature of the invention lies in the fact that the solid or hollow microspheres are intended to receive one or more pharmaceutical, alimentary or chemical active principles. The process according to the invention allows these active principles, dissolved or dispersed in the organic solvent soluble in the aqueous phase, to be incorporated in the emulsion droplets then, in proportion to the dissolution of the solvent, to constitute one of the constituent elements of the co-precipitate with the matrix polymer. The active principle, after drying, is thus included in the wall or the matrix of the microsphere where it is hidden from the physical or chemical agents.

The modification of the matrix can be achieved by varying the pH in the case where the polymer is sensitive to the pH, or simply by contact in aqueous medium in the case where the polymer is not sensitive to variations in the pH, or by varying the concentrations of the matrix polymer.

This process makes it possible to obtain determined release rates for the active principles, to obtain pre-determined homogeneous particle sizes, and by selecting the solubilization medium, to obtain a controlled released quantity of active principle.

The invention relates to the use of the microspheres obtained according to the process of the invention for the incorporation of active principles with a bitter or unpleasant taste. The invention relates in particular to the use of solid microspheres for the incorporation of medicinal active principles with an unpleasant or bitter taste, for obtaining tasteless galenic forms. Among the active products having an unpleasant, bitter or burning taste, mention may be made for example of quinine and its salts, chloramphenicol, thiamphenicol, ibuprofen, N-acetylcysteine, zinc salts, alkaline metal glutamates and paracetamol.

The invention also relates to the use of microspheres obtained according to the invention for the incorporation of dyes or sweetening products in order to produce a form of powdered, industrial or alimentary use, and thus a more convenient one.

The invention further relates to the use of microspheres according to the invention as a form with controlled release of active principles.

The invention offers the advantage of not requiring the use of chlorinated or aromatic solvent such as chloroform or methylene chloride, which removes the toxicological and ecological risks linked to the use and disposal of such a solvent.

The invention also offers the advantage of permitting the recovery and reutilisation of the hydroalkanol phase containing a part or the totality of the surfactant, in such a way that use can be made of a smaller volume of solvent and a smaller quantity of waste water containing organic products is thus discharged.

Within the scope of the present application, the term “disperse” relates to the operation of putting into solution or putting into suspension according to the degree of solubility of the active product.

### EXAMPLE I

Production of Hollow Microspheres Containing Ibuprofen as the Active Substance

In this process, Eudragit S 100 marketed by the company Röhm Pharma is chosen as the constituent polymer of the particles insoluble with an acid pH. As the surfactant, use is made of Span 60 which is a non-ionic surfactant.

The centesimal composition of the formula used for the production of the hollow microspheres is as follows (in dry product):

| | |
|---|---|
| Eudragit S 100 | 14.01% by mass |
| Ibuprofen | 82.26% by mass |
| Span 60 | 3.73% by mass |

The concentration in dry matter is around 1.7% by mass in a solvent mixture constituted by around 2.9% of ethanol and 97.1% of water.

The Eudragit S 100 and the ibuprofen are solubilised at room temperature for 15 minutes using magnetic stirring in ethanol.

The surfactant is solubilised in the presence of magnetic stirring at 80 C. for 20 minutes in a fraction of the water, then it is added to the rest of the aqueous phase in the working receptacle.

After dissolution of each of the components, the ethanol solution is gradually added to the aqueous solution, while stirring at 1700 revs/min. with the aid of an Ystral mixer at room temperature. After completing the addition, the stirring speed is reduced to 300 revs/min. and continued for 30 minutes.

At the end of this time, the particles are recovered by filtration under vacuum and the obtained microspheres are washed 3 times in water then dried at 50 C. for 24 hours in a ventilated plate oven.

### EXAMPLE II

Production of Solid Microspheres Containing Ibuprofen as the Active Substance

A mixture of two pH-independent esterified acrylic polymers, carrying a quaternary ammonium group, is chosen as the constituent polymer of the matrix. It is a mixture of Eudragit RL 100 and Eudragit RS 100 both marketed by the company Rohm Pharma.

The surfactant used is a non-ionic polyoxyethylene sorbitan marketed under the name Span 60.

The centesimal composition in dry product of the formulation intended for the production of the solid microspheres is as follows:

| | |
|---|---|
| Eudragit RS 100 | 10.95% by mass |
| Eudragit RL 100 | 14.60% by mass |
| Ibuprofen | 68.61% by mass |
| Span 60 | 5.84% by mass |

The concentration in dry matter is around 1. 8% by mass in a solvent mixture constituted by around 1.9% of ethanol and 98.1% of water.

Ibuprofen and the Eudragit RS 100 and RL 100 are put into solution under magnetic stirring at room temperature, in ethanol.

The surfactant is solubilised under magnetic stirring at 80 C. for 20 minutes in a fraction of water, then added to the rest of the aqueous phase in the working container.

After complete dissolution of all the ingredients, the organic solution is gradually added to the aqueous solution under strong stirring (3000 revs/min.) with the aid of an Ystral mixer.

Once the addition has been achieved, the stirring speed is reduced to 1200 revs/min. and the stirring is continued for 30 minutes.

At the end of this period, the particles are recovered by filtration under vacuum The solid microspheres are washed 3 times in water then dried at 50 C. for 24 hours in a ventilated plate oven.

### EXAMPLE III

Scale-up

The process of example I and example II can be used to produce microspheres on a scale 8 times higher than that used for the laboratory tests. The results obtained by increasing the quantities of the ingredients and solvents in the corresponding proportions allow one to presume a possible industrialisation of the process on an even larger scale.

So it has been possible to transpose the process of producing hollow microspheres on a scale 40 times larger than that of the laboratory tests.

### EXAMPLE IV

Recycling of the Aqueous Phase

The process according to the invention makes it possible on the industrial scale to recover an aqueous phase at the time of filtration or during the washing, which also contains at least a part of the water-soluble surfactant. It is thus possible to achieve a considerable saving on water and to avoid discharging large quantities of waste water in the waste disposal systems.

### EXAMPLE V

Production of Microspheres of Ibuprofen with Eudragit S 100

The procedure is that according to the technology described in French patent application 97 13155. The desired aim is to study the effects of a reduction in the granulometry achieved by working at high speed. It is possible to work without distinction in solution in alcohol at 96 (BG) or in absolute alcohol. It is possible to use Tween 80 or SPAN 60.

a) Use of Simethicone

An emulsion exhibiting a fine granulometry is thus obtained without the particles that have not completely solidified becoming deformed or breaking. The dissolution time for the microspheres obtained is around 3 hours at pH =6.8.

On the other hand, the washing times for the particles are lengthened and the recovery is difficult.

b) Increasing the Stirring, Incorporation and Temperature Parameters

Among all the tests carried out, whose results are very satisfactory, it is found that the temperature is an important factor and plays a role in the size of the internal cavity.

c) Scale Transposition to 750 ml, 4 l, 40 l and 270 l of Aqueous Phase

The process is thus completely operational whatever the scale is.

The duration of the introduction of the organic phase is 1 min 30, stirring is at 1200 rpm The duration of the experiment is 5 min. Very hollow particles are obtained if the experiment is carried out at 25 C. “Quasi-solid” particles are obtained if the experiment is carried out at 40 C. (temperature of aqueous phase). These particles can be advantageously used after compression for producing tablets with instantaneous disintegration called Flashtab®, according to the technique described in French patent 2.679.451.

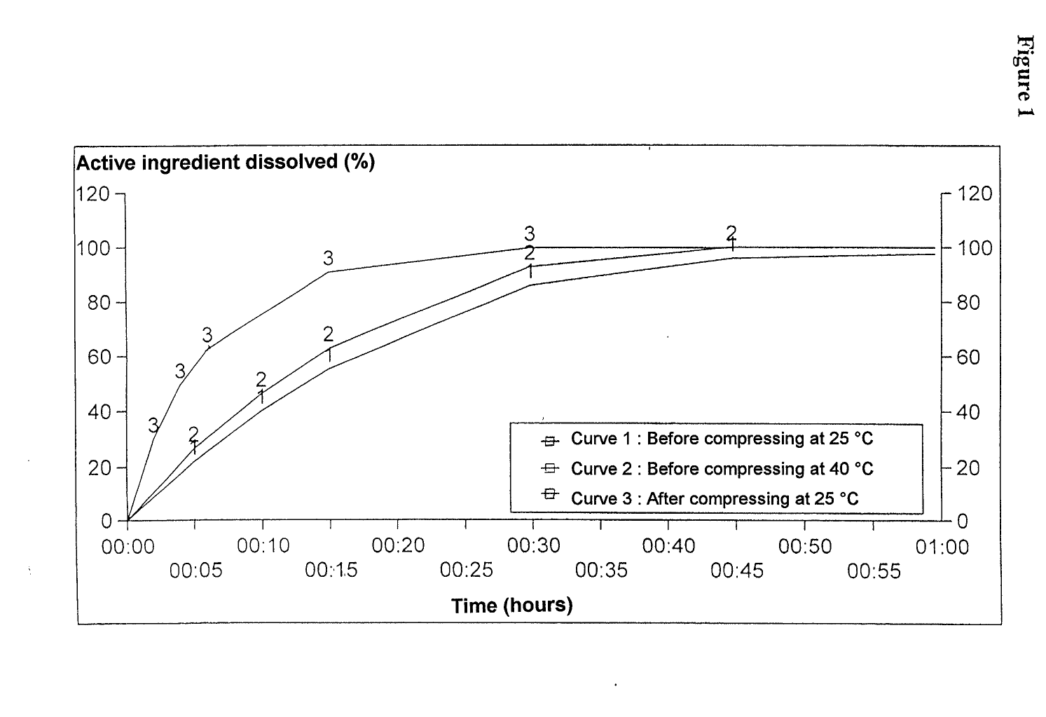

FIG. 1 describes the dissolution profile of the microspheres at 25 C. and at 40 C. Also represented is the dissolution profile of the microspheres after compression of those obtained at 25 C.

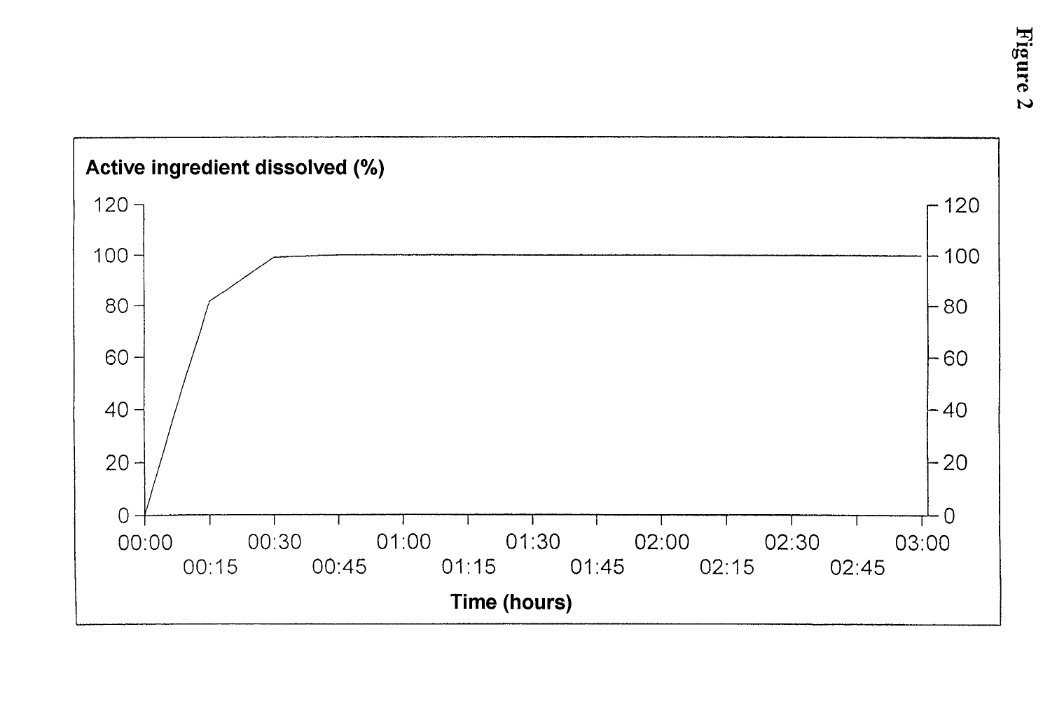

FIG. 2 shows the dissolution profile of the finest particles obtained in an operating unit of 40 l of aqueous phase.

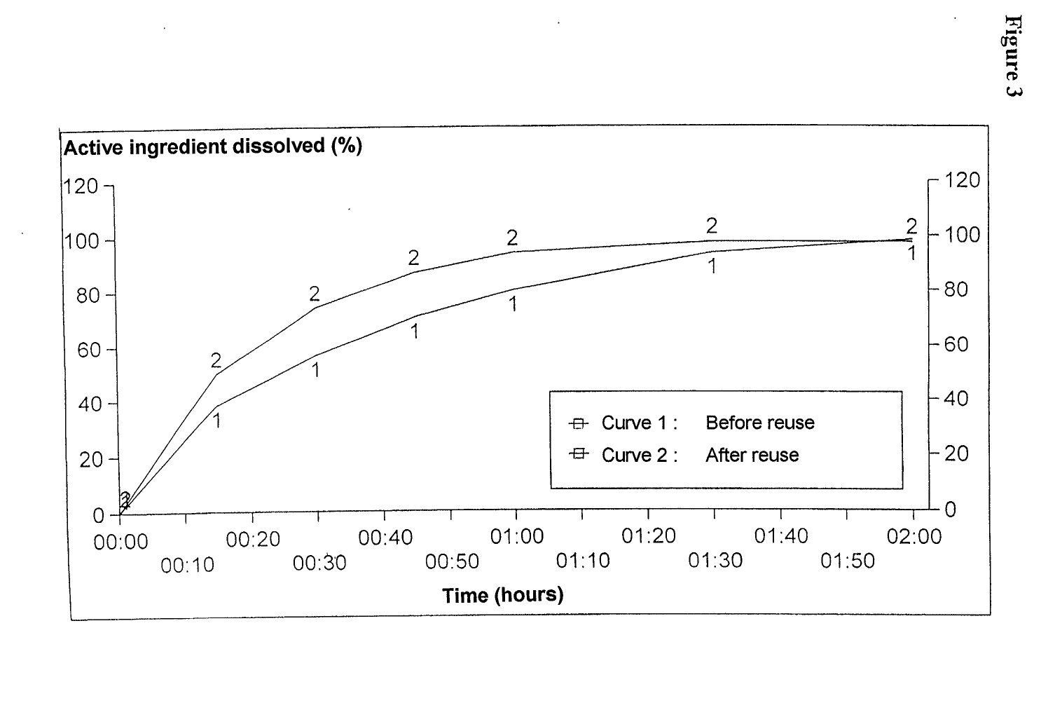

FIG. 3 represents an example of the reuse of the aqueous phase in this system.

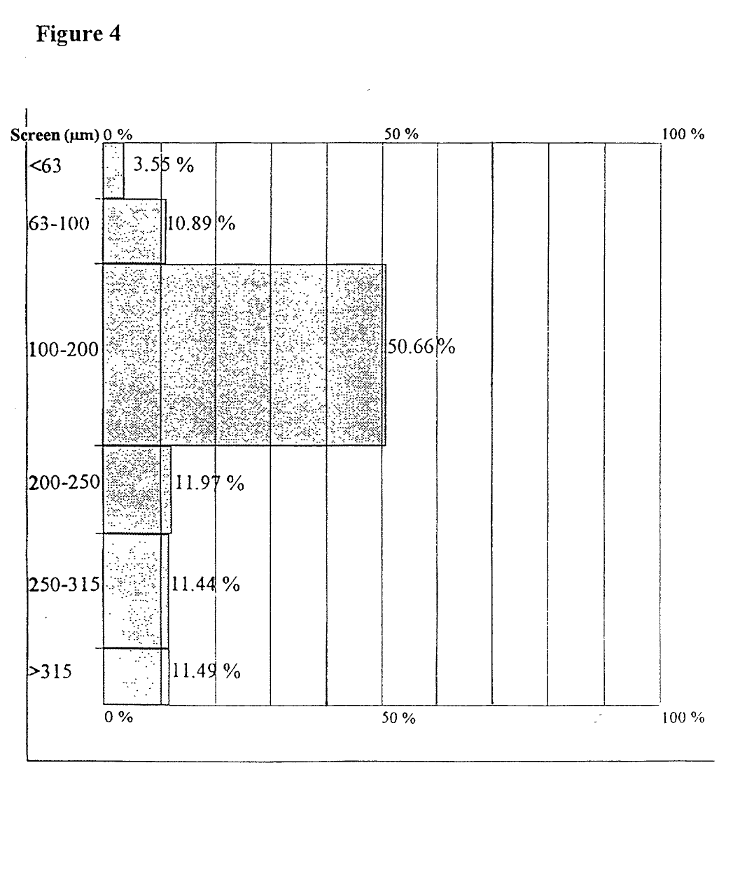

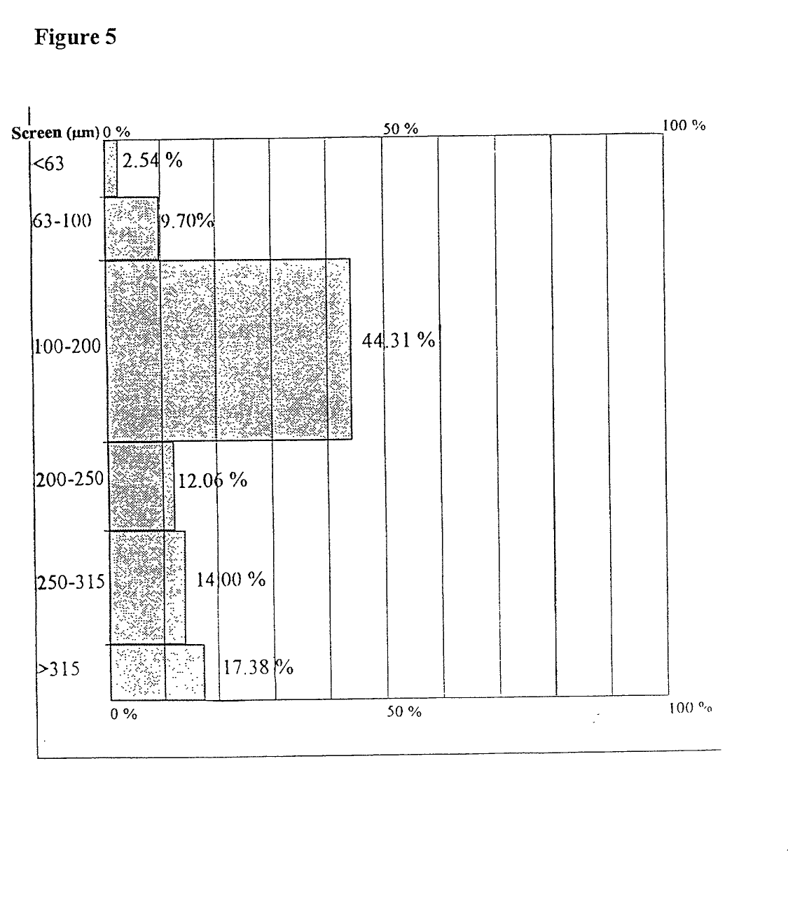

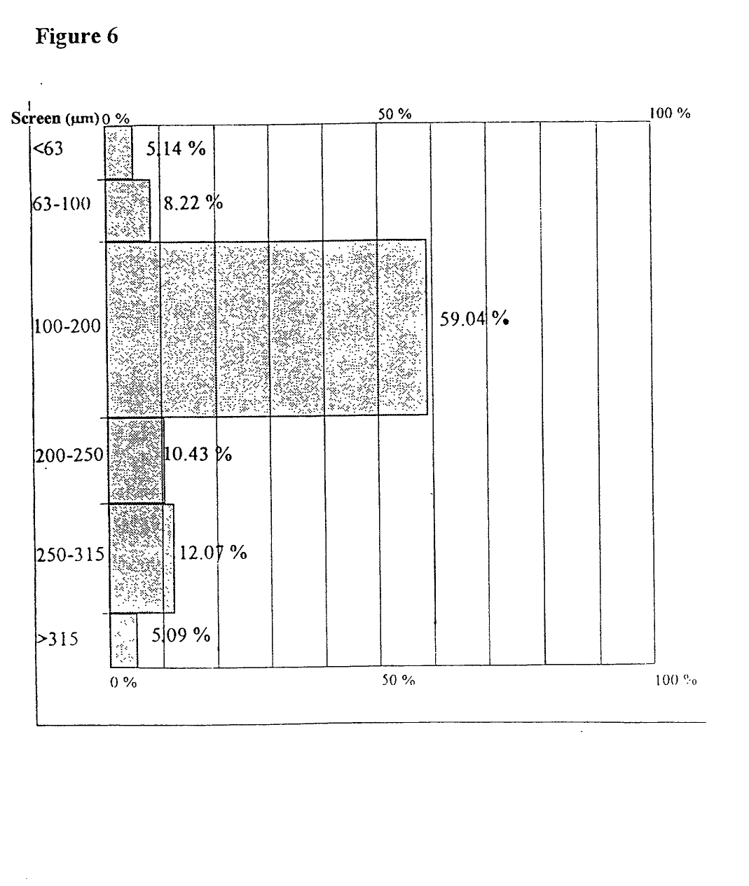

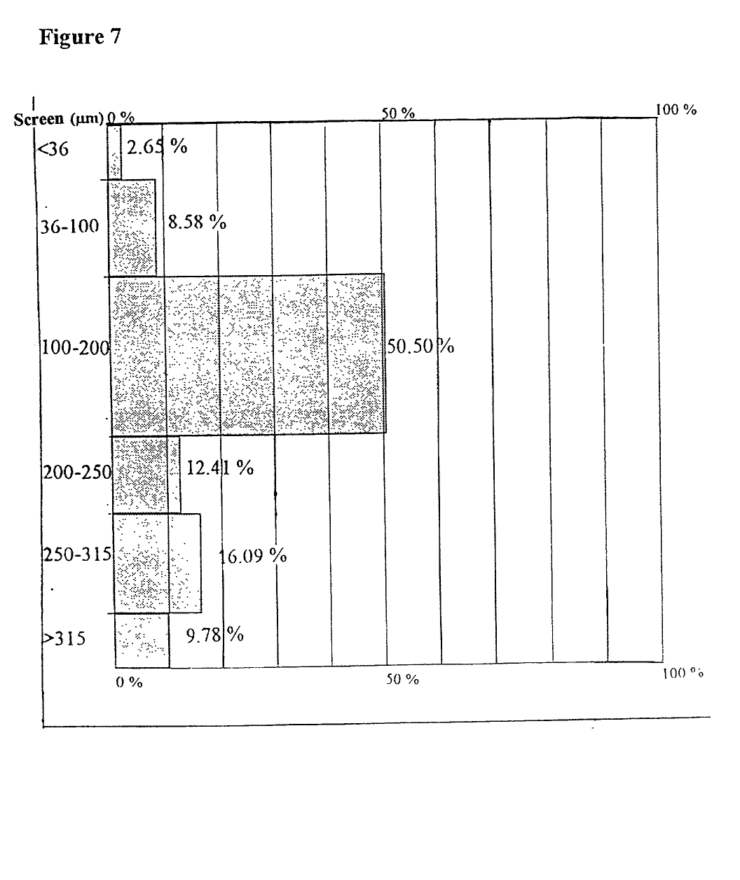

It was demonstrated, moreover, that it is possible to modify the granulometric distribution by acting on the physical parameters, and this in a reproducible manner (FIGS. 4, 5, 6 and 7—repro 1 and 2, same parameters: repro 3 and 4, same parameters).

### EXAMPLE VI

Production of Microspheres of Ibuprofen with Eudragit RS 100 and Eudragit RL 100

Study of the Influence of the Solvent

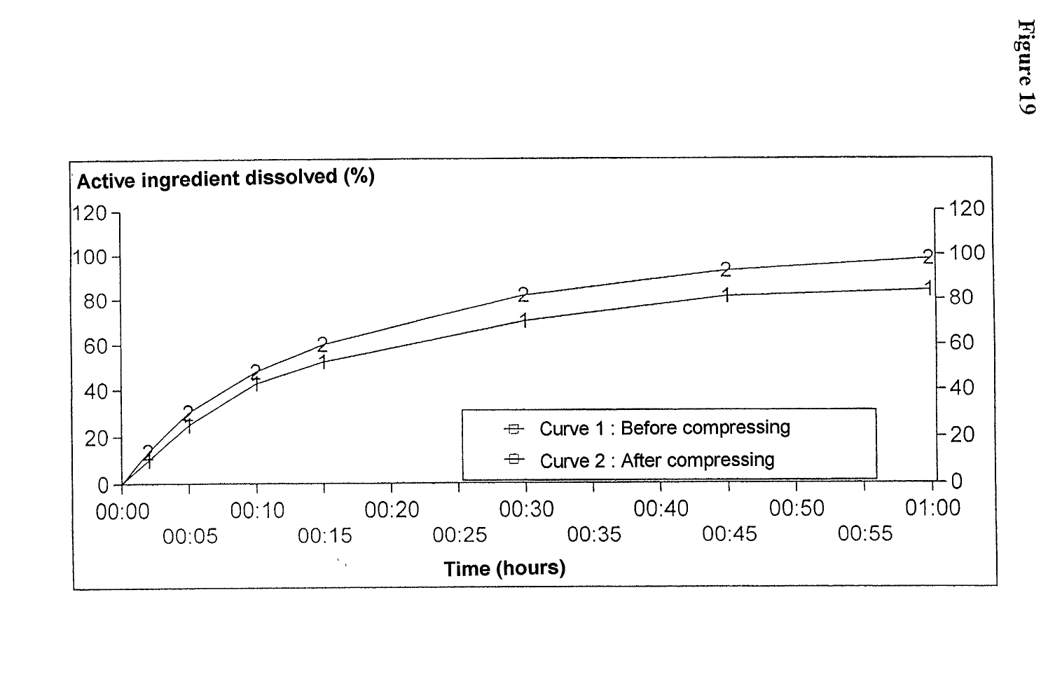

An attempt was made to study the widest possible range of solvents essentially to verify whether it would be possible to obtain very fine granulometries (95%<315 pm) and whether changes would be found in the dissolution times. These tests showed that with absolute ethanol, 96 BG alcohol and with isopropanol, almost the same particles sizes were obtained. These particles were able to be incorporated in tablets of the Flashtab® type (FIG. 19).

### EXAMPLE VII

Production of Microspheres of Ketoprofen with Eudragit S 100

The aim is to produce hollow microparticles of ketoprofen by studying the influence of the pH as well as the yield and the stability of the emulsion.

It is found that the addition of 0.28 g (2.65%) of Aerosil R972 in the organic phase markedly reduces the proportion of the aggregates. The pH exerts an influence on the yield and on the number of desorganized crystals.

The rate and depth of incorporation as well as the continuity of the stirring are essential parameters for optimising the stability of the emulsion.

The optimisation of the system of surfactants was also studied. It is thus possible to use a sucro-ester such as the product CRODESTA F-70 in a dose of 3.56 g/l, Tween 81 in a dose of 14.23 g/l in the organic phase and SPAN in a dose of 1.2 g/l in the aqueous phase.

The pH of the aqueous phase is adjusted to 8.9 by 0.01 N sodium hydroxyde. The incorporation of the organic phase into the aqueous phase takes place in 1 to 2 minutes. Stirring is at about 500 r/min. The duration of the experiment is 40 minutes.

The percentage by mass of dry matter (excluding surfactant) of Eudragit S 100 is 14.16% and that of ketoprofen is 83.19% one also operates with liquid phases constituted of between 3.6 and 7.2% of ethanol. Hollow particles are thus obtained with a yield higher than 80%.

### EXAMPLE VIII

Production of Microspheres of Ketoprofen with Eudragit RS 100 and Eudragit RL 100

The aim is to obtain solid particles. Particles having a regular shape are obtained with a good yield. On the other hand, the masking of the taste is not perfect.

The volume of water used is 750 ml; the volume of ethanol is 20 ml; the mass of Eudragit RL 100 is 2 g; the mass of Eudragit RS 100 is 1.5 g; the mass of ketoprofen incorporated is 12.5 g. 0.9 g of SPAN 60 is added to the aqueous phase and 0.7 g of CRODESTA F-70 is added to the organic phase. Incorporation at the surface takes place in 4 minutes by stirring at 1500 revs/min. The duration of the experiment is 45 minutes.

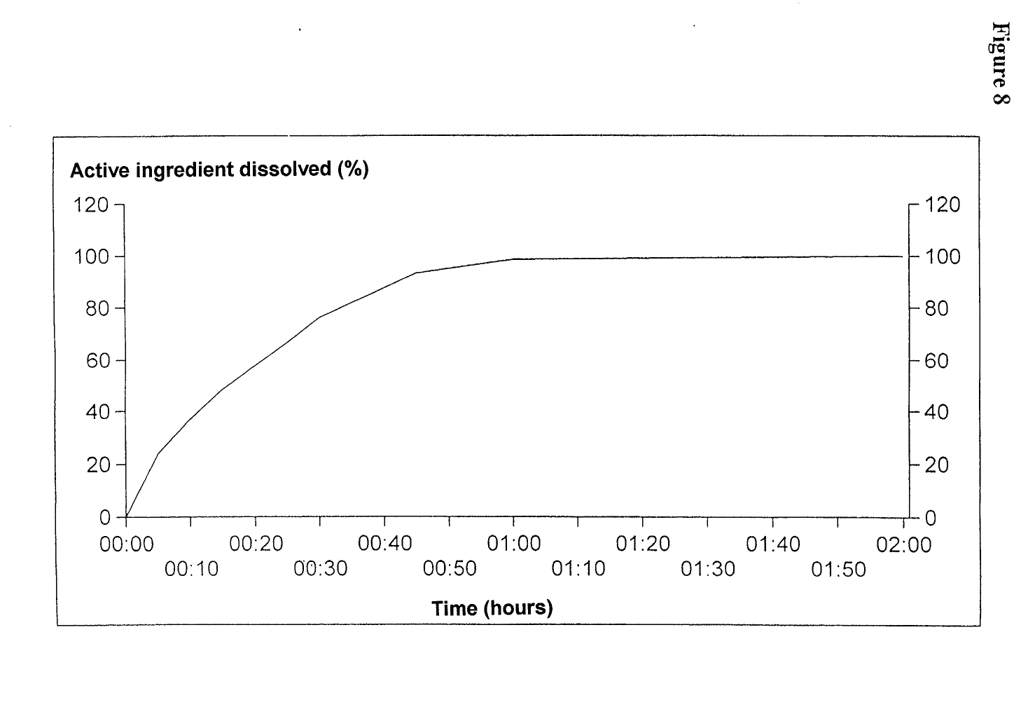

The operating unit was raised to 6 l of aqueous phase without encountering the slightest problem FIG. 8 shows the corresponding dissolution profile.

### EXAMPLE IX

Production of Microspheres of Aspirin with Eudragit S 100

In a first test, the procedure involved supersaturating the organic phase (28.1 ml of ethanol) with aspirin. Hollow microspheres are obtained in the following manner:

| | | |
|---|---|---|
| volume of water: | 750 | ml |
| volume of ethanol: | 28.1 | ml |
| CRODESTA F-70 in the organic phase: | 0.1 | g |
| SPAN 60 in the aqueous phase: | 0.9 | g |
| mass of Eudragit S 100: | 1.5 | g |
| mass of ASA: | 16.31 | g |
| pH = 10.3 | | |
| surface incorporation in 1 minute | | |
| stirring: | 1 100 | rpm |
| duration of experiment: | 20 | min. |
| temperature of the organic solution: | >60 | C. |

In a second test, a thickening agent was added to overcome the supersaturation of the organic phase. Explotab (4% p/p) or sodium alginate is added. The problem is to study the ease of recovery of the microspheres and to observe the size of the cavities. Another test was carried out with other Eudragits (Eudragits RS 100, RL 100). Solid particles of aspirin were thus obtained.

| | | |
|---|---|---|
| mass of RL 100: | 2 | g |
| mass of RS 100: | 1.5 | g |
| mass of SPAN 60 in the aqueous phase: | 0.9 | g |
| mass of ASA: | 16.31 | g |
| surface incorporation in 20 seconds | | |
| stirring at 500 rpm during the introduction then at 1000 rpm | | |
| duration of experiment: | 20 | min. |
| temperature of organic solution: | >60 | C. |

### EXAMPLE X

Production of Microspheres of Ibuprofen with Ethyl Cellulose

Use is made of ethyl cellulose N7NF, or N22NF or N50NF.

The aim is to produce particles having a cavity as regular as possible.

M=mass of polymer +mass of active ingredient

%P=percentage of polymer

v=volume of ethanol

1) Hollowest Possible Particles

| | |
|---|---|
| volume of water: | 750 ml |
| M/v = | 571 mg/ml |
| % P = | 25% |
| M = | 12 g, 24 g, 36 g |
| surfactant (Span 60): | 0.9 g, 1.8 g, 2.7 g |
| pH of the aqueous phase buffered at 10.1 by | 0.01 N sodium hydroxide |
| stirring: | 2 000 rpm |
| incorporation in solution in 30 seconds | |
| so-called low stirring position | |

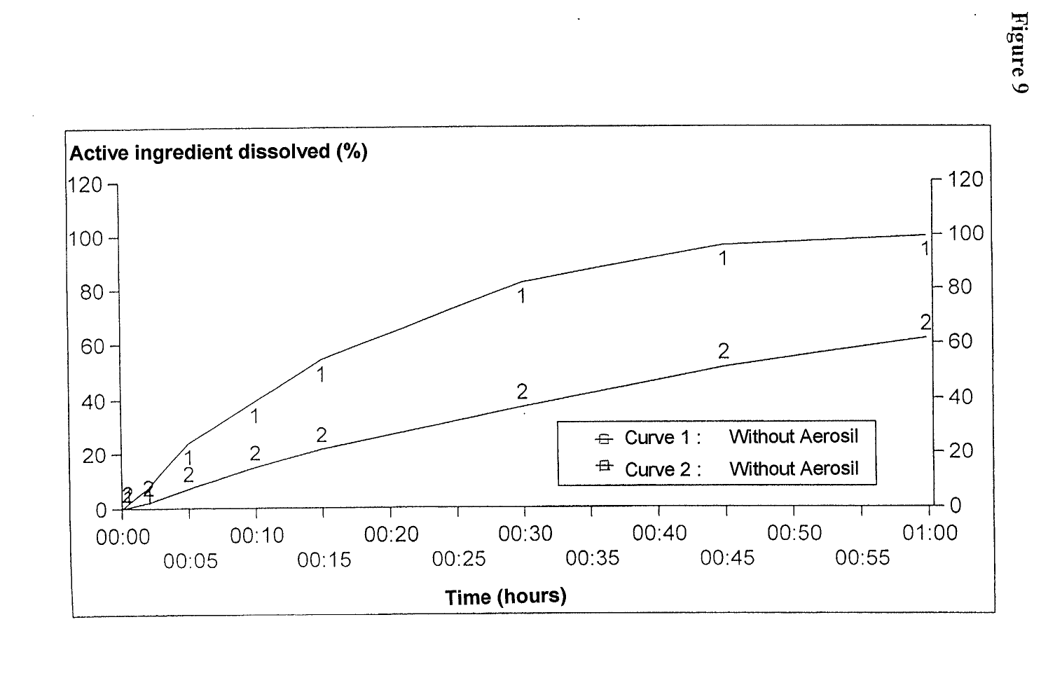

FIG. 9: the stripping of Aerosil slows down the dissolution profile of the hollow particles.

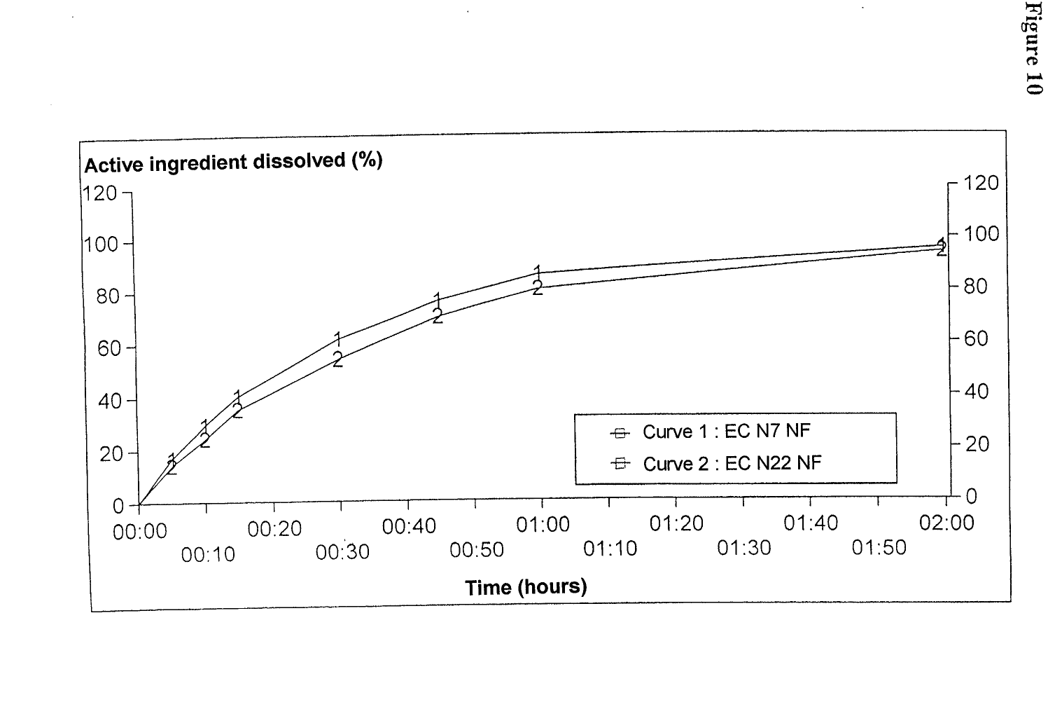

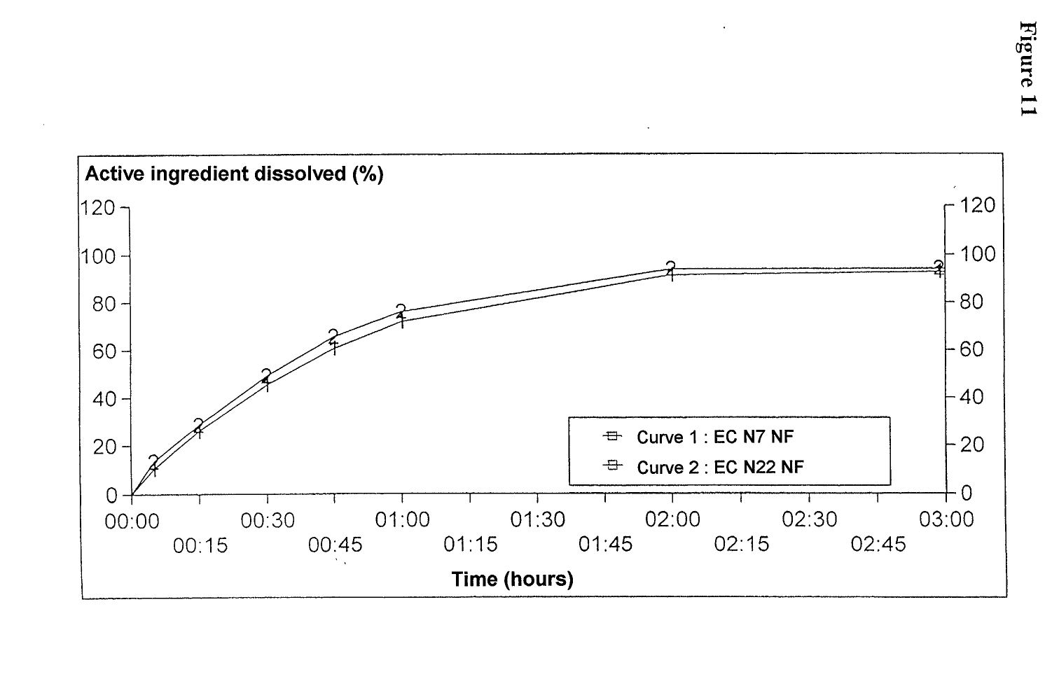

FIGS. 10 and 11: use of different qualities of ethyl cellulose at two dissolution pH.

2) Selected Solid Particles

| | |
|---|---|
| volume of water: | 750 ml |
| M/v = | 328 mg/ml |
| % P = | 4.3% |
| M = | 9.2 g 18.4 g |
| surfactant (SPAN 60): | 0.9 g 1.8 g |
| pH of the aqueous phase buffered at 10.1 by | 0.01 N sodium hydroxide |
| stirring: | 1 500 rpm |
| incorporation in the solution in 1 minute | |
| so-called INTERMEDIATE stirring position | |
| possibility of incorporation after compression | |
| in Flashtab tablets | |

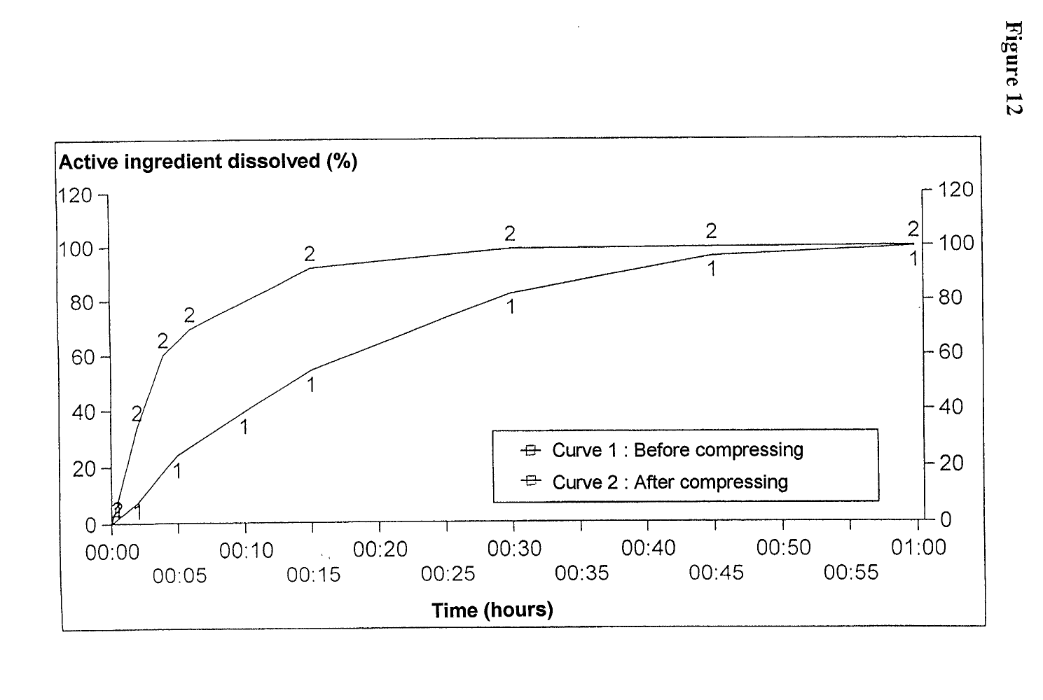

FIG. 12: dissolution profiles of the microspheres before and after incorporation in Flashtab) tablets

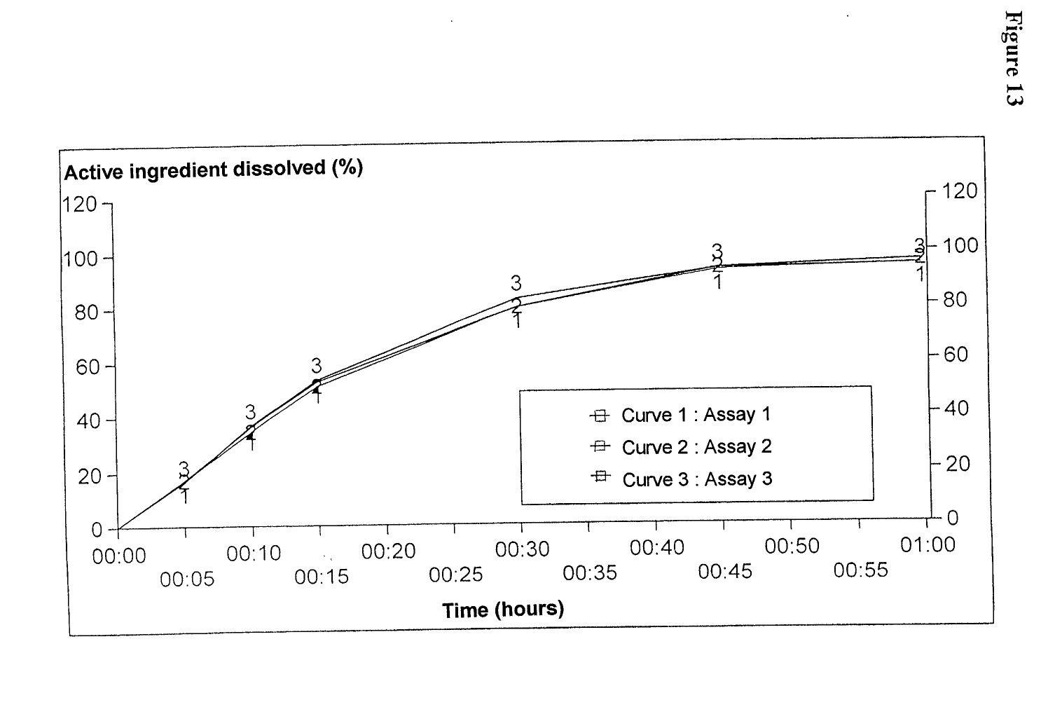

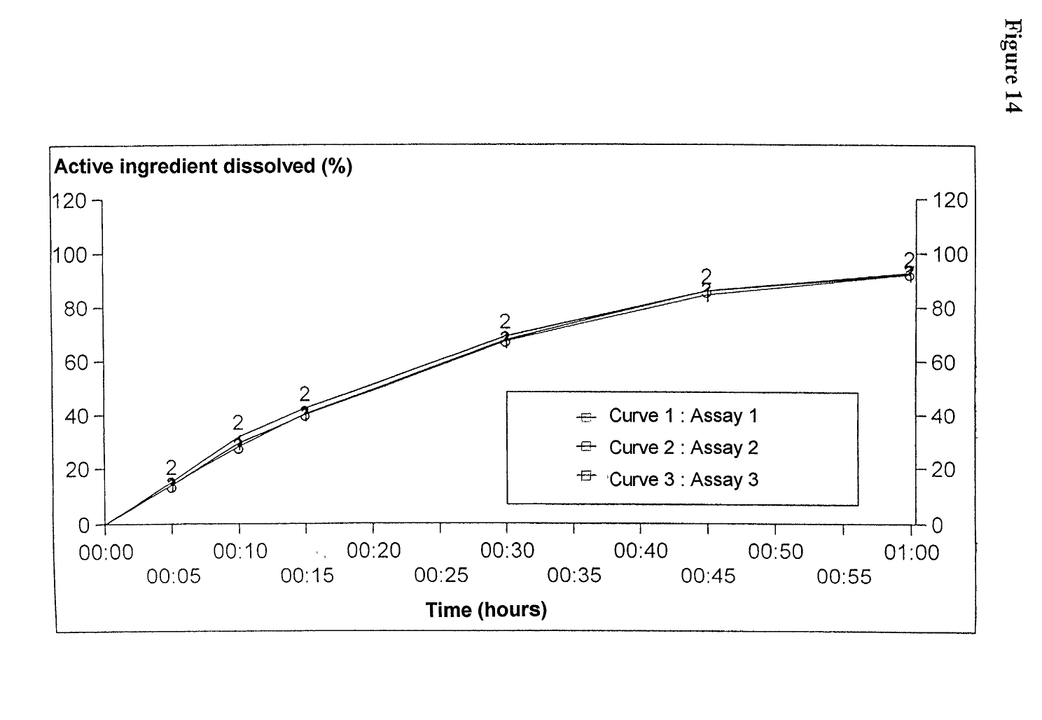

FIGS. 13 and 14: dissolution profiles in the batches obtained by means of 750 ml and 270 l of aqueous phase

Granulometry: no. 4 P86

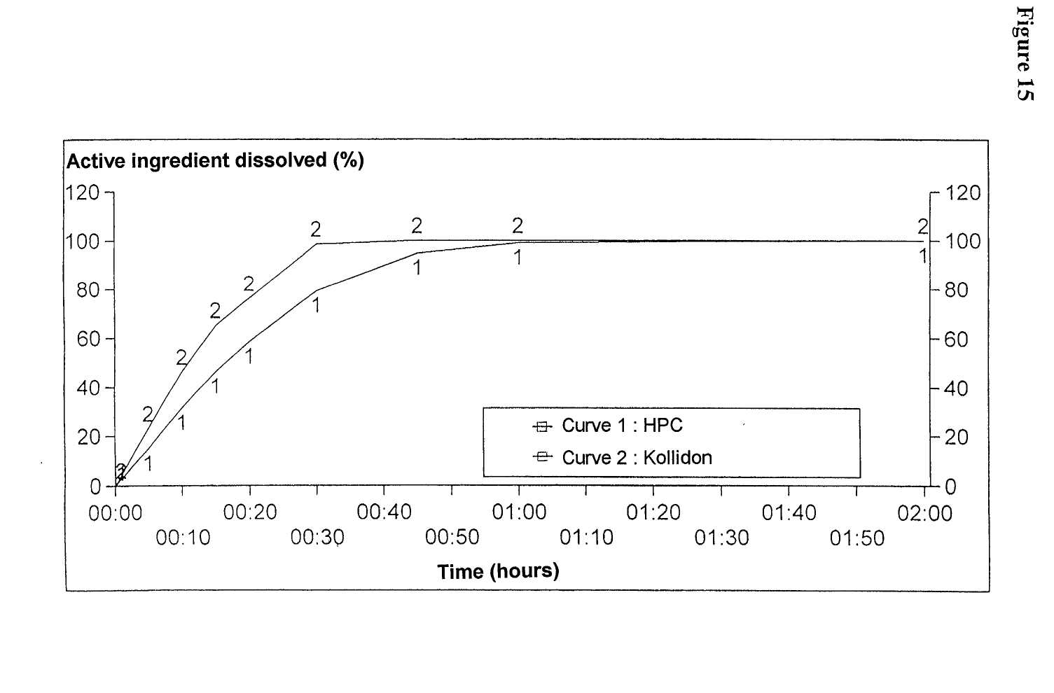

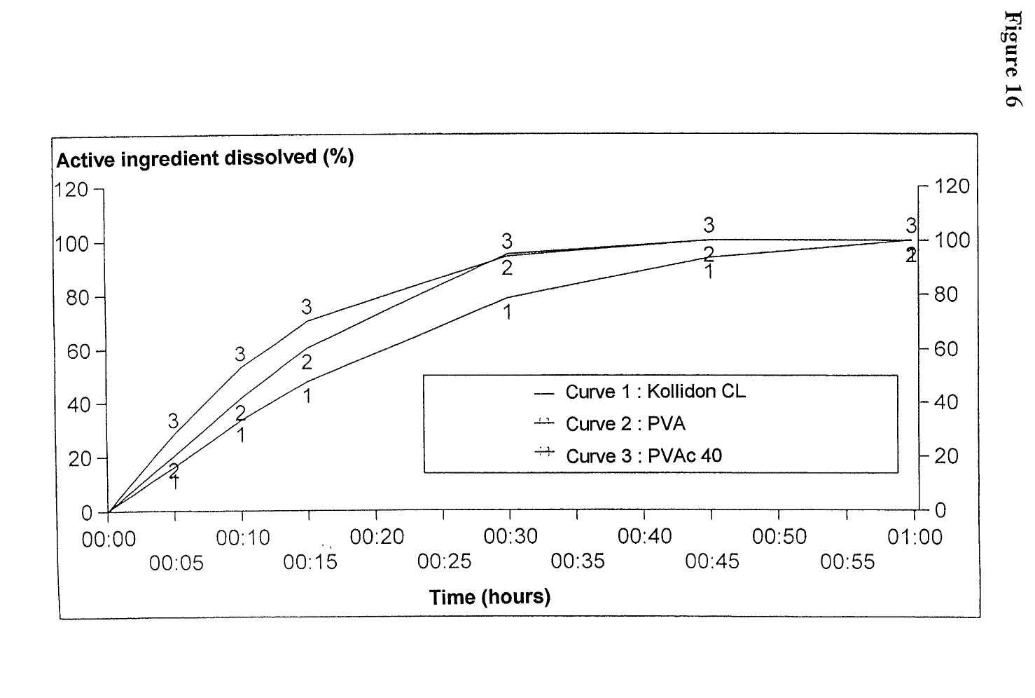

FIGS. 15 and 16 relate to other polymers.

### EXAMPLE XI

Coating of Microspheres

This operation was carried out on solid microparticles obtained on the basis of example VI, and on hollow microparticles obtained on the basis of example I.

This coating is carried out in a fluidised air bed apparatus.

The size of the microspheres is between 250 and 500 μm

The coating suspension is composed of a mixture of Eudragit L30D and Eudragit NE30D, plasticised by Eudraflex and supplemented by the addition of an inert agent of the talc type.

Following a bottom spray, the coating with 25% dry matter performed on the solid particles is gastro-resistant. These solid particles, once compacted, retain their gastro-resistant characteristics.

### EXAMPLE XII

Incorporation of an Oil

In this process, Eudragit S 100 is chosen as the matrix polymer. As the surfactant, use is made of CRODESTA F-70 which is a sucro-ester.

Centesimal composition of the formula in dry product:

| | |
|---|---|
| Eudragit S 100: | 13.91% by mass |
| Ibuprofen: | 81.74% by mass |
| CRODESTA F-70 | 4.35% by mass |

The concentration is around 0.6% by mass in a mixture of solvents made of about 1.31% of Mygliol 810 N, 0.26% of ethanol and 98.43% of water.

The incorporation is slow made at the surface, and it is carried out while stirring at 1000 rpm which will be lowered at the end of the incorporation.

After 10 minutes, Aerosil R972 is added at the surface of the aqueous phase and the particles are recovered after 60 seconds.

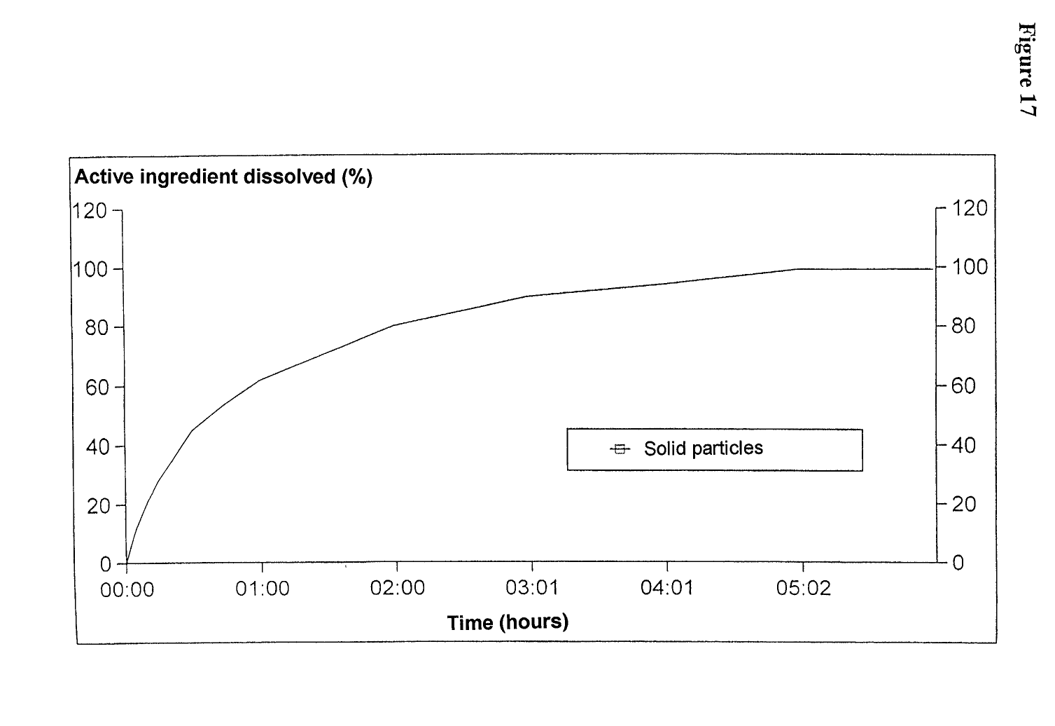

FIG. 17 shows the dissolution profile obtained at pH =7.2.

Examples V to IX permit to confirm the validity of the model developed according to example X in which:

an increase in the proportion of ethanol in the formulation, whatever the M/v ratio, reduces the degree of porosity of the solid particles;

an increase in the proportion of polymers with constant M/v contributes towards creating the internal cavity of the hollow particles;

there is an M/v ratio equal to approx. 0.57 mg/ml which permits hollow microspheres to be obtained in the optimum manner. If this ratio is departed from, the proportion of hollow particles obtained diminishes;

the stirring speed only exerts an influence on the final granulometry.

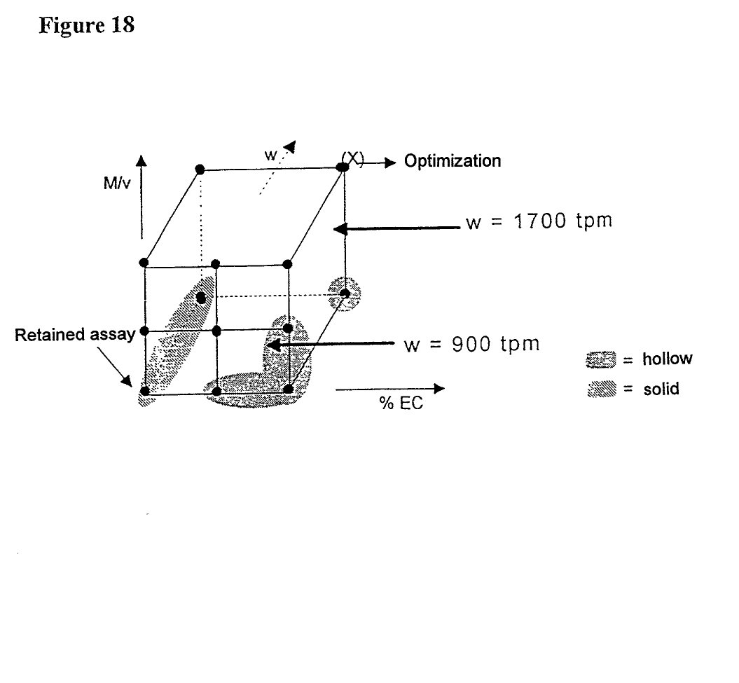

FIG. 18 shows the influence of the M/v ratio (dry matter/volume of ethanol), the percentage of polymer (% of ethyl cellulose) and the stirring speed (w) on the nature of the particles obtained.

The dissolution tests showed that the microspheres thus prepared dissolved completely in 3 hours at pH 7.2. The microspheres obtained are more or less hollow.

Additional tests led to the preparation of solid microparticles by lowering the porosity. The particles dissolve to the extent of 80% in 25 minutes.

Examples V to IX made it possible to establish in particular that the ratio between the mass of dry matter and the volume of ethanol varies in the same way as the porosity of the particles, that the increase in the percentage of ethyl cellulose in the formula leads to an increase in the volume the cavity, that the stirring speed exerts an influence on the granulometry of the particles and that the coating with an inert material such as Aerosil 972 modifies the dissolution profile in a very significant manner.

The value of the pH plays a certain role in the rate of dissolution of microspheres containing an ionic product.

The operating scale was gradually increased from 750 ml, 4 l, 40 l and 270 l.

The process is perfectly capable of industrialisation.

TABLE I
Summary of the systems studied permitting hollow particles of ibuprofen
to be obtained

| | Ethyl | Eudragit | Hydroxypropyl | |
|---|---|---|---|---|
| | cellulose | S 100 | cellulose (HPC) | Kollidon V |
| Sample | 1 | 2 | 3 | 4 |
| Theoretical | 750 | 855 | 855 | 855 |
| titre (mg/g) | | | | |
| Calculated titre | 700 | 850 | 888 | 941 |
| (mg/g) | | | | |
| Process time | 10 | 4 or 15 | 5 | 5 |
| (min) | | | | |
| Yield (%) | >90% | >90% | >80% | >90% |
| Size of charge | 9 | 9; 500 and | 8 | 9 |
| (g) | | 7000 | | |

TABLE II
Summary of the systems studied permitting solid particles of ibuprofen to be obtained

| | | | Polyvinyl | Polyvinyl | |
|---|---|---|---|---|---|
| | | Eudragit | acetate | alcohol | Kollidon |
| | Ethyl cellulose | s RS, RL | (PVAc 40) | (PVA) | CL |
| Sample | 5 | 6 | 7 | 8 | 9 |
| Theoretical titre (mg/g) | 953 | 720 | 855 | 855 | 855 |
| Calculated titre (mg/g) | 975 | 730 | 827 | 898 | 770 |
| Process time (min) | 8 | 30 | 5 | 5 | 5 |
| Yield (%) | >90% | >90% | >90% | >90% | >80% |
| Masking of taste | good | average | correct | average | correct |
| Size of batches (g) | 9.3; 500 and | 9 | 9 | 9 | 8 |
| | 3500 | | | | |

TABLE III
Summary of other systems studied permitting hollow particles to be
obtained

| | Ketoprofen/ | |
|---|---|---|
| | Eudragit S 100 | Aspirin/Eudragit S 100 |
| Sample | 10 | 11 |
| Theoretical titre (mg/g) | 832 | 915.9 |
| Process time (min) | 40 | 20 |
| Yield (%) | >85.8% | >80% |
| Size of batch (g) | 9 | 8 |

TABLE IV
Summary of other systems studied permitting solid particles to be obtained

| | Ketoprofen/ | |
|---|---|---|
| | Eudragits RS, RL | Aspirin/Eudragits RS, RL |
| Sample | 12 | 13 |
| Theoretical titre (mg/g) | 781 | 823 |
| Process time (min) | 35 | 20 |
| Yield (%) | >90% | >80% |
| Size of batch (g) | 9 | 8 |

1. Process for obtaining microspheres containing a pharmaceutical, alimentary or chemical active ingredient, which consists in dispersing one or more matrix-forming agents in a hydroalkanolic solution containing the active ingredient or ingredients in the presence of a surfactant in such a way as to form an O/W emulsion of said matrix-forming agent, in allowing the alkanol to diffuse from the emulsion droplets towards the continuous phase under stirring, so as to form in the droplets a co-precipitate of matrix-forming agent and active ingredient, in separating them from the continuous phase, possibly in washing them with a suitable solvent and in drying them so as to recover microspheres.

2. Process for obtaining solid microspheres according to claim 1, in which one or more matrix-forming agents and an active ingredient are dispersed in an alkanolic solvent, in the presence of one or more surfactants, so as to form with the aqueous phase an O/W emulsion distributed in a continuous aqueous phase, the alkanol is allowed to diffuse towards the aqueous phase to ensure solidification of the wall of the droplet, then the microspheres are separated, the washing and drying performed and solid microspheres are recovered.

3. Process according to claim 1 and claim 2, in which the matrix agent permitting solid spheres to be obtained is chosen among the ethyl celluloses, the acrylic acid polymers, polyvinyl acetate, polyvinyl alcohol and the polyvinyl pyrrolidones.

4. Process according to claims 1 and 2, in which the matrix-forming agent of the acrylic type is an acrylic ester polymer carrying a quaternary ammonium group having the formula:

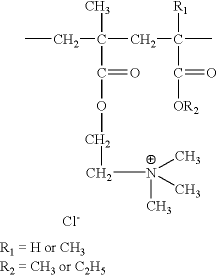

5. Process according to claims 1 to 4, in which the matrix-forming agent of the acrylic type is one of those marketed under the names Eudragit RS 100, Eudragit RL, Eudragit S 100, Eudragit L 100-55 and Eudragit RS-PM.

6. Process for obtaining hollow microspheres according to claim 1, in which one or more matrix-forming agents and an active ingredient are dispersed in an alkanolic solvent, in the presence of one or more surfactants, so as to form with the aqueous phase an O/W emulsion distributed in a continuous aqueous phase, the alkanol is allowed to diffuse towards the aqueous phase to ensure solidification of the wall of the droplet, then the microspheres are separated, the washing and drying are performed and hollow microspheres are recovered.

7. Process according to claim 6, in which the matrix agent is an ionic or non-ionic acrylic ester polymer, a polymer of the cellulose type, a polyoxyethylene or polyoxypropylene, a copolymer of the EVA type or a vinyl pyrrolidone polymer.

8. Process according to any one of claims 2 and 6, in which the matrix-forming agent of the acrylic type is one of those marketed under the names Eudragit RS, Eudragit S, Eudragit RL, Eudragit L and Eudragit E and Eudragit NE.

9. Process according to any one of claims 2 and 6, in which the matrix agent of the cellulose type is selected among ethyl cellulose, hydroxypropyl cellulose and hydroxy-propylmethyl cellulose.

10. Process for obtaining microspheres according to any one of claims 1 to 9, in which the stirring of the medium, the temperature of the aqueous and alkanolic phases, the time of incorporation of the alkanolic phase in the aqueous phase and the stirring position in the reaction medium are regulated in such a way as to form essentially solid microspheres or hollow microspheres.

11. Process according to any one of claims 1 to 9, in which at least two matrix-forming agents are associated so as to obtain solid, small and compact microspheres.

12. Process according to claim 1, in which the microspheres undergo a coating with an inert diluting agent or with a film-forming agent.

13. Process according to claim 1, in which a dispersible inert diluting agent is added to the aqueous phase so as to modify the texture of the external wall.

14. Process according to claim 12 or 13, in which the inert diluting agent is a colloidal silica such as Aerosil or a silicon such as simethicone.

15. Process according to claim 2, in which the alkanol is ethanol.

16. Process according to any one of the preceding claims, in which the proportion of alkanol in the reaction medium is increased so as to diminish the porosity of the solid microspheres.

17. Process according to any one of the preceding claims, in which the ratio between the quantity of matrix-forming polymer (M) and the volume of alkanol (v) determines or plays an important role in the formation of an internal cavity in the microspheres.

18. Process according to claims 6 and 9, in which the ratio M/v is of the order of 0.57 mg/ml so as to obtain hollow microspheres in the optimum manner, in the case of ethyl cellulose.

19. Process according to any one of the preceding claims, in which the content of surfactant ranges from 0 to 20% by weight of the dry product in the aqueous phase.

20. Process according to any one of the preceding claims, in which the content of surfactant ranges from 0 to 40% by weight in the organic solvent.

21. Process according to any one of the preceding claims, in which the content of active ingredient incorporated in the micro spheres ranges from 1 to 99% of the dry product.

22. Process according to any one of the preceding claims, in which the content of matrix-forming polymer incorporated in the microspheres ranges from 1 to 99% of the dry product.

23. Process according to claim 8, in which the microspheres exhibit a granulometry determined by the stirring speed, which ranges from 10 to 1000 μm.

24. Process according to any one of the preceding claims, in which the microspheres can, after compression, be incorporated in tablets dispersible in the mouth with immediate release.

25. The solid microspheres obtained according to the process of any one of claims 1 to 23.

26. The hollow microspheres obtained according to the process of any one of claims 1 to 23.

27. Use of the solid or hollow microspheres obtained according to the process of any one of claims 1 to 9 for the incorporation and taste-masking of substances with a bitter or unpleasant taste.

28. Use of the solid or hollow microspheres obtained according to the process of any one of claims 1 to 9 for producing pharmaceutical compositions with a controlled release of the active ingredient.

29. Use of the solid or hollow microspheres obtained according to the process of any one of claims 1 to 9 for producing alimentary compositions incorporating a sweetening product or a dying product.

30. Use of the solid or hollow microspheres obtained according to the process of any one of the preceding claims for producing pharmaceutical compositions with improved bioavailability.

31. Process according to any one of the preceding claims in which the hydroalkanolic phase is recycled.
